(19) United States
(12) Patent Application Publication
(10) Pub. No.: US 2026/0357168 A1
(43) Pub. Date: Nov. 12, 2026

(54) MODULAR ADJUSTABLE BIOREACTOR FOR DECELLULARIZATION AND CELL SEEDING

(71) Applicant: RESEARCH INSTITUTE AT NATIONWIDE CHILDREN'S HOSPITAL, Columbus, OH (US)

(72) Inventors: Tendy CHIANG, Columbus, OH (US); Woo Yul BYUN, Columbus, OH (US); Lumei LIU, Columbus, OH (US)

(21) Appl. No.: 19/129,678

(22) PCT Filed: Nov. 15, 2023
(86) PCT No.: PCT/US2023/079747

§ 371 Date: May 14, 2025

Related U.S. Application Data
(60) Provisional application No. 63/384,028, filed on Nov. 16, 2022.

(51) Int. Cl. C12M 3/00; A61F 2/04; C12M 1/12

(52) U.S. Cl. CPC C12M 21/08; A61F 2/04; C12M 23/06; A61F 2002/046

(57) ABSTRACT

An adjustable, modular bioreactor has been developed for both partial or full decellularization of organs or tissues and recellularization of the decellularized organs or tissues. The allows for the use of different end pieces to secure different diameters of the tissue to be treated, and is adjustable in length, for example, using a slidable chamber housing, which can be shortened or lengthened merely by sliding of an endpiece, where the chamber remains sealed through the use of an O-ring or other seal and the endpiece is secured in position with a clamp.

### CROSS-REFERENCE TO RELATED APPLICATIONS

This application claims the benefit of and priority to U.S. Provisional Application No. 63/384,028 filed Nov. 16, 2022, the entire content of which is incorporated herein by reference for all purpose in its entirety.

### U.S. GOVERNMENT SUPPORT

This invention was made with government support under R01HL157039 awarded by the National Institutes of Health. The government has certain rights in the invention.

### FIELD OF THE INVENTION

This invention is generally in the field of bioreactors, specifically a single bioreactor for preparation of tissue derived graft to culture cells and tissues.

### BACKGROUND OF THE INVENTION

Bioreactors have been in use for a number of years to culture cells and tissues under conditions that allow for attachment, proliferation and differentiation into structures mimicking those present in nature. Bioreactors are tailored to specific organs and an understanding of developmental biology, including specific chemical, mechanical, and electrical stimuli, is needed to optimize bioreactor performance to enhance the function of each engineered organ or tissue. The premise of bioartificial organs is to strip an organ that is non-transplantable of its cellular components using a process termed decellularization to yield a scaffold on which to develop a new organ. See for example, Bijonowski Curr Opin Chem Eng. 2013 Feb. 1; 2(1):32-40. Important properties of these scaffolds are retention of native tissue architecture and maintenance of extracellular matrix (ECM) components and growth factors for proper cellular organization and differentiation. Scaffolds are then seeded with autologous or allogeneic cells to repopulate the matrix and return function to the organ.

The process of perfusion decellularization to produce a biological scaffold containing the structural proteins of an organ or tissue is well characterized for small animal models such as rodents. Physical and chemical methods have been used to remove cells and leave an intact ECM. The amount of DNA remaining within the ECM is typically used as a surrogate to measure efficiency of cell removal, and depends upon the cellular and extracellular composition of the organ or tissue, its geometry (planar or three dimensional) and method used. Organs are placed within containers designed for decellularization, allowing the organ to be perfused with solutions through its vasculature or submerged within fluid that is agitated by a stirrer or rocker. Decellularization is typically carried out at room temperature, but occasionally scaffolds are cooled to 4 °C to enhance ECM preservation or warmed to 37 °C when using enzymatic methods and/or detergents such as sodium dodecyl sulfate (SDS). As the organ decellularizes, cell fragments are washed from the scaffold and debris may build up within the decellularization bioreactor. In addition, decellularization agents may be diluted or sequestered, making this process less efficient. This is a factor in the need for bioreactors with continuous/controllable flow.

Several methods are used to reseed scaffolds. Dynamic methods tend to be more effective than static cell seeding. The most common dynamic method for reseeding is to directly add cells at high concentration into the vascular perfusion line just upstream of the organ, allowing cells to travel directly through the vascular tree into the scaffold and parenchyma. This method is universally used to recellularize the vasculature of hearts, lungs, and livers. Many investigators have delivered cells to an organ parenchyma through the scaffold vasculature; the cells are thought to traverse the vascular lining through holes or pores created by the decellularization process. A second method to reseed organ parenchyma is to inoculate cells into the bulk media and allow them to recycle through the circuit to reseed the organ, but this achieves a lower seeding efficiency (69%) compared to the multi-step process. Endothelial and organ parenchymal cells may be seeded together in a mixture or via separate inoculations of pure cell populations.

It is an object of the present invention to provide a single bioreactor for decellularization and reseeding and/or proliferation of cells on and in the decellularized tissue.

It is a further object of the present invention to provide a bioreactor for partial decellularization and reseeding of decellularized tissue not requiring removal or repositioning of the tissue.

It is another object of the present invention to provide a bioreactor that can be used with a wide range of organ/tissue dimensions and geometries, by providing adjustable and/or modular components.

It is another object of the present invention to provide a bioreactor that can be used with a wide range of organ/tissues/materials due to its modularity.

It is another object of the present invention to enable automation of precise media flow control, including concentration, flow rate, and sequence.

It is a still further object of the present invention to provide a bioreactor what can be physically manipulated to modify the reseeding conditions.

### SUMMARY OF THE INVENTION

Bioreactors have been developed for regenerative medicine where they are used for full or partial decellularization of organs and/or recellularization of decellularized organs. The process of partial decellularization requires the preservation of cell viability of select populations while removing other populations. A bioreactor has been designed to provide for full or partial decellularization of the tissue to be used as a scaffold for tissue regeneration, as well as culture and/or seeding of cells or tissue onto the decellularized scaffold. The perfused medium and the flow parameter and sequences determine the extent of decellularization.

The bioreactor can be used with a wide range of organ/tissues/materials due to its modularity and adjustability. The design enables automation of precise media flow control. The bioreactor can be physically manipulated to modify the reseeding conditions, including temperature, exposure to various wavelengths and regimens of light exposure. The design enables physical manipulation of the reseeding conditions (e.g., rotational reseeding through inoculation of bulk media). The system allows for sequential and/or simultaneous different media to be introduced into the interior of the graft from that on the exterior. Air-liquid interface cultures are possible by pumping in air instead of liquid in one of the inlets. Gases other than air, or air containing, particulate matter such as smoke or aerosolized compounds can be used when the device is used to screen for an effect of the media on the cells or the effect on the cells in the presence of a therapeutic agent to be screened.

The bioreactor has three principal components, an inlet end piece (referred to as a “plug”), including on the outside two ports, one in the center for a fluid such as a decellularization fluid, and one towards the outside of the tissue culture connection portion for introduction of a fluid such as cell culture medium and on the inside a connector for the tissue to be processed, wherein the decellularization media passes into the interior of the connected tissue and out through an outlet port on the outlet end piece (referred to as a“insert”), and the cell culture media passes into the bioreactor through an inlet port on the inlet connector to the outside of the tissue in the bioreactor, where it perfuses though the scaffold and then out the same outlet as the decellularization media.

The bioreactor has been designed to be readily secured to the tissue to be decellularized and reseeded, requiring minimal manipulation of the tissue during processing in the bioreactor. The bioreactor has an inlet port that delivers decellularization fluid into the interior of the tissue to decellularize, with a second inlet port in the same end piece that delivers fluid and cells to the outside of the tissue for recellularization, so that the tissue does not require removal or repositioning for the two processing steps. An outlet port is used to remove the decellularization fluid and cell culture media. The end pieces used to secure the tissue can be adjusted for length and diameter to accommodate additional types of tissue, not just trachea, but other tubular structures such as blood vessels, organs such as stomach and bladder, and discrete structures such as heart valves.

The end pieces can also be used to secure the arterial and venous blood supplies or ducts of various whole organs such as the kidney, liver lobes, and pancreas. Furthermore, if perfusion within an organ is unneeded, such as in the case for the processing of small volumes of nervous or adipose tissue, then unneeded ports can be closed for single-lumen flow.

Using different end pieces enables the processing of laminar tissues, materials, or organs such as skin or neural tissues. For example, end pieces that contain an adjustable support structure can be used to turn the laminar tissues, materials, and organs into a tubular structure which can then be clamped into place. In another embodiment, the endpiece design has spokes that serve as a scaffold for flat structures, such as a piece of skin. The skin is “draped” over the spokes and wrapped around the spokes, similar to how yarn is rolled onto a wire spool. The seam where the skin overlaps is held in-place by a built-in clip arm. The edges are secured using clips, wire, or glue.

These components can be interchanged to alter diameters and/or lengths or slid from within one piece to extend the length of the pieces, or vice versa. In a preferred embodiment, the effective chamber housing can be shortened or lengthened by sliding one of the end pieces into or out of the housing chamber, where it remains fluidically sealed by an O-ring or other fluid seal.

### BRIEF DESCRIPTION OF THE DRAWINGS

FIGS. 1A-1C are cross-sectional drawings of the flow paths of the bioreactor. FIG. 1A shows the drainage outlet insert 12 which is connected to the tissue scaffold 14, secured in place with a c-clip or retainer 16, and fluidically sealed with O-ring 18. FIG. 1B is the drainage outlet insert 12 in the tubular housing 20 showing the drainage outlet 22 from the tissue scaffold 14 and the drainage outlet 24 from the exterior of the tissue scaffold 14. FIG. 1C is the inlet plug 30 showing the fluid paths into the tissue 32 and to the exterior of the tissue 34, the inlet plug insert 36 into the tissue scaffold 14 and the O-rings 38 fluidically sealing the inlet plug insert 36 into the tubular housing 20.

FIGS. 2A-2D are cross-sectional views of the assembly of the bioreactor shown in FIGS. 1A-1C. FIGS. 2A and 2B show the clip 16, tubular housing 20, into which at one end is positioned the drainage outlet insert 12, wherein the clip 16 is positioned between 40 and 42, and the inlet plug 30 is positioned at the other end of the tubular housing 20, where O-rings 44a, 44b, 44c fluidically seal the tissue scaffold area within the tubular housing 20. FIG. 2C and FIG. 2D show the components of FIG. 2A and FIG. 2B assembled. FIG. 2D shows the assembled bioreactor in cross-section. Fluid flows through fluid channel 32 into the tissue scaffold (not shown) and out through fluid channel 22. Fluid flows into the tubular housing 20 through fluid channel 34 into the exterior space from the tissue scaffold and out port 48.

FIGS. 3A-3E are a second embodiment of the inlet plug for use with a larger diameter tissue scaffold. FIG. 3A is the c-clip or retainer 16. FIG. 3B shows tubular housing 20. FIG. 3C is the drainage outlet insert 50, having a larger diameter end 62 which secures the tissue scaffold, while end 52 is secured to the outside of tube 20 with the c-clip or retainer 16 at section 54. Section 56 and 58 are positioned within tubular housing 20. Fluid flow channel 64 runs from the tissue scaffold interior (not shown) out of the tubular housing 20. FIG. 3D is a cross-sectional perspective view of the drainage outlet insert 50. FIG. 3E shows the inlet plug 70 with larger diameter section 62 and 64, used to secure the tissue scaffold. O-rings at 66 and 68 seal the fluid within the tubular housing 20. Fluid flow channel runs from 66 through the inlet plug 70 into the tissue scaffold. Fluid channel 68 runs through the inlet plug 70 into the exterior of the tissue scaffold.

FIG. 4 shows the assembled components of the bioreactor for use with a larger diameter tissue scaffold shown in FIGS. 3A-3E.

FIGS. 5A-5D show the drainage outlet insert 80 for use with flat tissue rolled to form a tubular structure. FIG. 5A is a cross-sectional view of one of the plates 100 located on the end portion of drainage outlet insert 80 which secures one end of the flat tissue structure. Fluid hole 104 drains fluid from the tissue scaffold. FIG. 5B shows the top or inner most plate 100 having holes 102a-h inside rim 104. Tissue clip 108 secures flat tissue into a rolled tubular scaffold. FIG. 5C is a side perspective view, showing plates 100a, 100b, 100c, section 90, expanded section 88, indented section 86, retainer section 84, and end piece 82. Fluid channel 92 runs the length of the drainage outlet insert 80. FIG. 5D is a cross-sectional perspective view.

FIGS. 6A-6B show the inlet plug 106 for use with flat tissue rolled to form a tubular structure. FIG. 6A is a side perspective view; FIG. 6B is a side perspective view. The inlet plug 106 includes exterior section 122 with base rim 124, O-ring recession 118b, separating region 116, O-ring recession 118a, section 114, base support 112, plates 110a, 110b, 110c support prongs 106a-e, and tissue sheet retaining clip 108. It is understood that support prongs 106a-e can be flat, round, rectangular, or other geometry, vary in number, spacing, and height, but must be capable of supporting the flat tissue rolled to form a tubular structure, which is retained in position using clip 108. Fluid channel 124 is shown in FIG. 6A.

FIGS. 7A-7B show the assembly of the flat tissue bioreactor. FIG. 7A shows the two parts of the flat tissue bioreactor which are fitted together.

FIG. 7B shows how support prongs 106a-e fit into the holes of 102a-h on plate 100 of the drainage outlet insert 80. FIG. 7C is a cross-section of the assembled flat tissue bioreactor.

FIG. 8 is a cross-sectional view of the assembled bioreactor containing decellularized trachea 70, connected via valving 80 to peristaltic pumps 82, 84, connected with tubing to SDS 86 flowing to the interior of the trachea 70 and cell culture media DMEM 88 flowing to the exterior of the trachea 70. It is understood that the same type of device could be used with other tissues or organs, and other media, including air.

FIG. 9 is a photograph of a trachea grown in the bioreactor.

FIG. 10 shows the bioreactor connected to tubing and pumps for growing the trachea of FIG. 9.

FIGS. 11A-11C shows the bioreactor design and setup: Cross-section of the bioreactor with mock tracheal segment (11A). The dotted arrows represent the intraluminal and extraluminal flow paths. The pumps are represented on the right with the rectangle representing flexible fluid valving and splitting. Rabbit trachea which has been affixed to the two modular end fittings of the bioreactor (11B). Fully installed bioreactor with connected tubing and media flow (11C).

FIGS. 12A-12D. Live/dead assay and viability quantification: Representative axial live/dead images of native and processed trachea. (12A-12C) Quantified viability demonstrating that conventional processing results in partially decellularized tracheal grafts with chondrocyte viability lower than that of native and bioreactor-processed trachea (12D). (*** denotes significance with P≤0.001)

FIGS. 13A-13E. Masson's trichrome: Representative axial images of native and processed trachea stained with Masson's trichrome. (13A, B, D) A representative cropped and stain-only image of (13B) processed using the AFAT algorithm. (13C) Stain percentages which demonstrate no differences between the groups (13E).

FIG. 14A-14B. Compression testing method and results: Example load-compression graph with images demonstrating correlated tracheal occlusion. (14A) The arrow shows the datum at 50% occlusion which was used to compare between groups. Force at 50% occlusion which demonstrates that conventional processing results in partially decellularized tracheal grafts that occlude at lower loads when compared to native and bioreactor-processed trachea. (14B) (* denotes significance with P≤0.05)

### DETAILED DESCRIPTION OF THE INVENTION

Bioreactors are most commonly used for cell culture. More rarely bioreactors have been developed for regenerative medicine where they are used for recellularization of decellularized organs. In most cases where the naturally produced tissue is used as a matrix for the tissue engineered materials, the cells and other immunogenic proteins are removed, then the remaining extracellular matrix is placed into a cell culture bioreactor, where cells are seeded and then grown on the extracellular matrix.

Less commonly due to the inherent difficulties associated with partial decellularization, the tissue is only partially decellularized, then the remaining cells and/or host cells, separated from the organ/tissue donor's original cells, re used to repopulate the decellularized tissue. The process of partial decellularization requires the preservation of cell viability of select populations while removing other populations. For example, the selective sparing of chondrocytes may preserve native cartilage and its physical properties, which is difficult to substitute using synthetic materials and processes. Alternatively, the bioreactor can be used for partial decellularization of tissue to remove immunogenic cell types while preserving immunoprotected (or immunoprivileged) cartilage. The graft is then able to support epithelial regeneration, by host cells, either pre or post implant, to form an implantable/implanted graft.

### I. DEFINITIONS

A bioreactor is an apparatus for growing cells, which provide culture media, temperature control, and exchange of gases to maintain growth.

Decellularization refers to a chemical or physical process for removing the cellular compartment of living tissues, creating an acellular extracellular matrix (ECM) scaffold that can support cellular attachment and growth. Decellularization employs detergents, salts, enzymes, and/or physical means to remove cells from tissues or organs while preserving the ECM composition, architecture, bioactivity, and mechanics. A plethora of decellularization methods exist for different applications, such as those in Gilbert, et al. (2006). Decellularization of tissues and organs. Biomaterials 27, 3675-3683; Badylak, S. F., Taylor, D., and Uygun, K. (2011) Annu. Rev. Biomed. Eng. 13, 27-53. Recellularization is the process where cells are reseeded onto a scaffold and/or cells that were not removed are allowed to reproliferate in the scaffold.

Epithelialization is a process of covering denuded epithelial surface. The cellular and molecular processes involved in initiation, maintenance, and completion of epithelialization are essential for successful wound closure.

Perfusion in cell culture is a process that uses a method to keep cells in a bioreactor while continuously exchanging culture medium. Fresh medium replenishes nutrients and carbon sources, while cellular waste and medium depleted of nutrients are removed.

An allograft is a tissue graft of cells from a donor of the same species as the recipient but not genetically identical. An autograft is a tissue graft of cells from one part to another of the same individual's body.

Immunogenicity or immunoreactivity results from the biomaterial being detected by the body's immune system as a foreign object.

Immunoreactive biomaterials, especially wear particles, are detected by antigenic reactions on cells. A biochemical cascade then occurs, whereby T-helper cells migrate towards the biomaterial. This immune response can result in rejection of the biomaterial, and non-union between the biomaterial and the wound site. For bio-integrative products, this would be a significant hindrance to successful functioning. Immunogenicity may also be brought about by biomaterials that have become damaged or oxidised and therefore no longer non-immunogenic.

Scaffolds, typically made of polymeric biomaterials or decellularized extracellular matrix or bone provide the structural support for cell attachment and subsequent tissue development.

### II. MODULAR BIOREACTOR COMPONENTS

There are a number of benefits of using a modular system.

The first is scalability This is achieved through the use of modular pieces, allowing for larger or smaller diameters, longer or shorter chambers (including chambers where two pieces are slidably engaged to change the length, larger or smaller inlet outlets for connection to tissue, and connectors that can be used to attached to tubular tissues or laminar tissue that is rolled into tubular structures for processing.

A second benefit is that the components can be individually serviced, separately, assembled, or in circuits.

A third benefit is that the device is easy to assemble and disassemble for cleaning, reconfiguration, and replacement of components, adding the benefit of lower cost for repairs, rather than having to replace the entire assembly. The bioreactor and connection components do not include screws or specialized clips, brackets, fasteners, and specialized gaskets that are costly and require tools to incorporate.

A fourth benefit is that there is a minimal number of parts, also going to economy and flexibility and adjustability, especially for affordable and quick fabrication, including many readily available components such as luer lock tips, gaskets and O-rings to seal slideably connected pieces, to allow for adjustment of length, or rapid replacement of parts with different diameters.

A fifth benefit is that the components can typically be manufactured using relatively inexpensive technologies such as extrusion and simple fabrication such as 3D printing. The design is made to be easily 3D printed, injection molded, or machined on a lathe or drill. Virtually any fabrication method, even from materials such as ceramic or glass, can be used, due to the lack of complex or enclosed geometry. More specific fitments may require more complex fabrication methods. O-rings can be directly assembled using off-the-shelf silicone, nitrile, fluorocarbon, neoprene, polyurethane, fluorosilicone, etc. O-rings. Otherwise, they may be directly included through additive manufacturing if the printer has those capabilities (multiple nozzle printers that extrude thermoplastic polyurethane and other materials such as liquid silicone rubber) such as Luer lock components, pumps, and sterile tubing which are found in most medical centers. The design also has no enclosed volumes or heat/chemical-sensitive fabrication required (e.g. adhesives or welding) so it can more easily be sterilized.

A sixth benefit is that reduced contamination results from the use of closed circulation, which is compatible with sterile Luer lock components such as syringes and manifolds. Everything can be assembled within a tissue culture hood, then connected to tubing. This means that the enclosed areas are in a separate sterile environment and can be moved from place to place without compromising sterility. This is unlike other bioreactors that often require assembly with openings to an incubator or manually fill or exchange media (due to no flow).

### Housing and Modular End Pieces

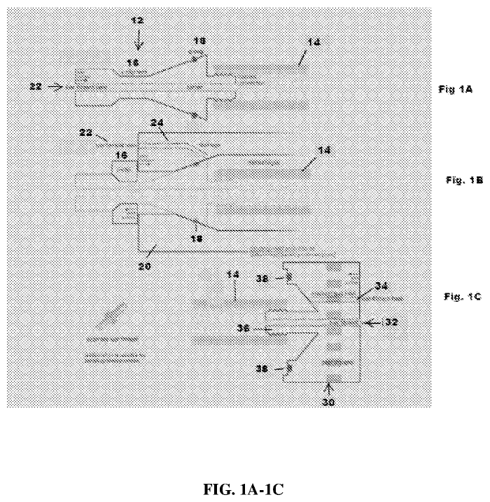

FIGS. 1A-1C are cross-sectional drawings of the flow paths of the bioreactor. FIG. 1A shows the drainage outlet insert 12 which is connected to the tissue scaffold 14, secured in place with a c-clip or retainer 16, and fluidically sealed with O-ring 18. FIG. 1B is the drainage outlet insert 12 in the tubular housing 20 showing the drainage outlet 22 from the tissue scaffold 14 and the drainage outlet 24 from the exterior of the tissue scaffold 14. FIG. 1C is the inlet plug 30 showing the fluid paths into the tissue 32 and to the exterior of the tissue 34, the inlet plug insert 36 into the tissue scaffold 14 and the O-rings 38 fluidically sealing the inlet plug insert 36 into the tubular housing 20.

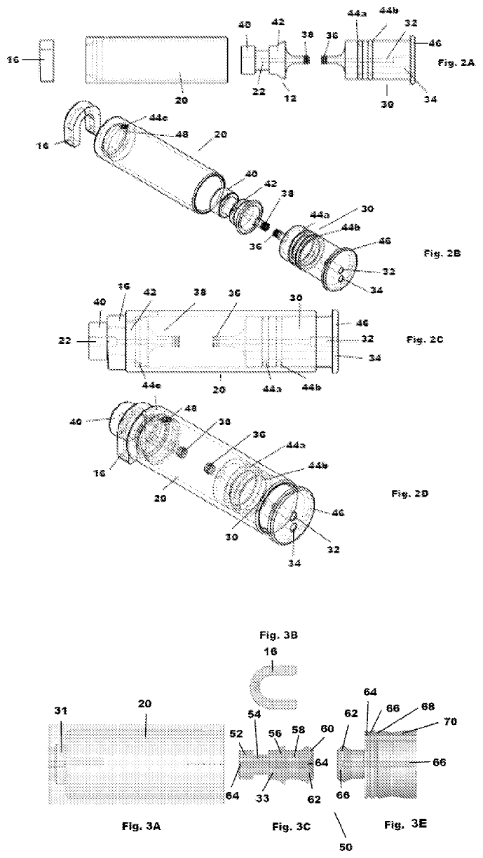

FIGS. 2A-2D are cross-sectional views of the assembly of the bioreactor shown in FIGS. 1A-1C. FIGS. 2A and 2B show the clip 16, tubular housing 20, into which at one end is positioned the drainage outlet insert 12, wherein the clip 16 is positioned between 40 and 42, and the inlet plug 30 is positioned at the other end of the tubular housing 20, where O-rings 44a, 44b, 44c fluidically seal the tissue scaffold area within the tubular housing 20. FIG. 2C and FIG. 2D show the components of FIG. 2A and FIG. 2B assembled. FIG. 2D shows the assembled bioreactor in cross-section. Fluid flows through fluid channel 32 into the tissue scaffold (not shown) and out through fluid channel 22. Fluid flows into the tubular housing 20 through fluid channel 34 into the exterior space from the tissue scaffold and out port 48.

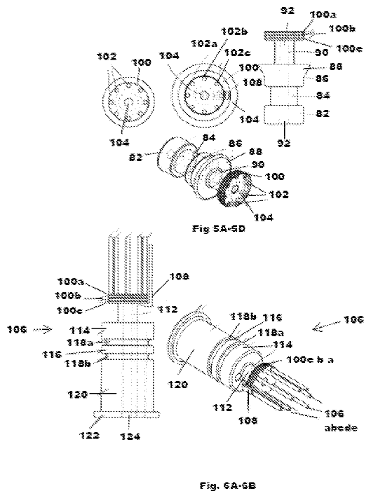

An advantage of the inlet and outlet pieces (FIG. 1, A-1C 20, 22; FIG. 2A 12, 30; FIG. 4, 50, 70) is that the length and/or diameter of the pieces used to secure the tissue to be decellularized and/or seeded can be modified for use with different types and/or sizes (diameter and length) of tissue.

FIGS. 2A-2D show a first embodiment including a smaller diameter piece to secure more narrow structures such as trachea and blood vessel, or structures from a small animal or infant.

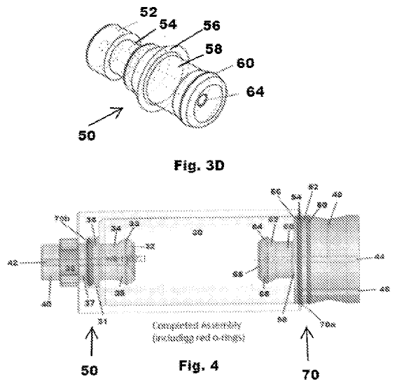

FIGS. 3A-3E are a second embodiment of the inlet plug for use with a larger diameter tissue scaffold such as intestine or bladder. FIG. 3A is the c-clip or retainer 16. FIG. 3B shows tubular housing 20. FIG. 3C is the drainage outlet insert 50, having a larger diameter end 62 which secures the tissue scaffold, while end 52 is secured to the outside of tube 20 with the c-clip or retainer 16 at section 54. Section 56 and 58 are positioned within tubular housing 20. Fluid flow channel 64 runs from the tissue scaffold interior (not shown) out of the tubular housing 20. FIG. 3D is a cross-sectional perspective view of the drainage outlet insert 50. FIG. 3E shows the inlet plug 70 with larger diameter section 62 and 64, used to secure the tissue scaffold. O-rings at 66 and 68 seal the fluid within the tubular housing 20. Fluid flow channel runs from 66 through the inlet plug 70 into the tissue scaffold. Fluid channel 68 runs through the inlet plug 70 into the exterior of the tissue scaffold.

FIG. 4 shows the assembled components of the bioreactor for use with a larger diameter tissue scaffold than shown in FIGS. 3A-3E.

In one embodiment the tubular structure is secured at each end, as shown in FIGS. 1-4, to form a fluid tight connection to the end pieces, so that fluid passes within or without the tissue. The tissue is secured by various means such as zip ties, wire or sutures, or clamps. Actual examples of the device for processing of trachea are shown in FIGS. 8 and 9.

Representative dimensions include the tubular housing which has an inner diameter of between about 5 mm to about 250 mm; the tubular housing which has an inner length of between about 10 mm to about 1 m; and the insert, cap, and plug which have an outer diameter of between about 5 mm to about 250 mm. Dimensions of openings in organs, tissues, and tissue grafts for connection have an outer diameter of between about 100 μm to about 50 mm and a length of between about 1 mm to about 50 mm.

### Clamps

As shown in FIGS. 1A-1C, the tissue 10 to be decellularized is connected to the drainage port 12 of the cylindrical reactor and to the inlet port 14 of the reactor using c-shaped retainers or clips 16, magnets, or other fixating methods such as elastic bands, suture, wire, zipties or adhesives such as a heat-sensitive adhesive that can form a liquid or gas seal.

The clamp component which fits within the external slot of the drainage outlet insert (for example, 22 in FIG. 2A) fixates internal components by means of friction, magnets, or adhesive and a scaffold. This clamp is used to adjust the length between the inlet and outlet end pieces so that it can accommodate different lengths of tissue scaffolds.

Tubing (not shown) is connected to the endpiece inlet 20 and outlet 22, preferably using a luer lock fitting or equivalent fitting. Gas and/or cell culture medium enters at 24 and is discharged through outlet 26. The tubular housing has one smooth/unfeatured opening enabling unidirectional insertion of internal components and adjustability of internal components (cap and insert with fitted tissue or organ).

### Sealing Means

O-rings or gaskets enable liquid and gas-tight sealing against the cap's central opening. O-rings/gaskets are typically formed of elastomers such as nitrile, neoprene, ethylene propylene (EPDM Rubber), silicone, fluorocarbon, and polytetrafluoroethylene (“PTFE”) manufactured by extrusion, compression molding, injection molding, transfer molding, or machining. O-rings/gaskets formed of thermoplastic polyurethane and other materials such as liquid silicone rubber can be manufactured by additive manufacturing and installed or directly integrated within the appropriate parts of the tubular body, cap, or plug.

O-rings 18, 44a, 44b prevent leakage.

In one embodiment, the tubular housing has an internal ledge enabling fixation of internal components (O-rings/gaskets and insert) (see, for example, FIG. 2B, 44c). The tubular housing's internal ledge and insert (see FIG. 2A, 42, for example) function as a boss seal, i.e., a conical surface that gradually expands the O-ring for water/gas-tight sealing as the insert is clamped, to form a liquid and gas-tight junction. The insert component has an external slot that couples with the clamp to consistently fixate the insert component within the tubular housing.

### Caps and Ports for Tubing and Wiring

The tubular housing may have additional openings for connections for liquid media, gas perfusion, and/or electrical wires. The insert component has an opening or multiple openings that serves as a connection for liquid media, gasses, or electrical wires into or around the fitments. The insert component can have features surrounding or within the fitments that enable fixation of scaffolds or structures outside or inside of the tissue/organ. The cap component has an opening or multiple openings that serve as a connection for liquid media, gasses, or electrical wires into the intraluminal chamber. The cap component may also have a fitment that enables fixation of one or more tubular organs/tissues/tissue scaffold in the form of a “barb” or structures that enable the fitment of non-tubular organs/tissues/tissue scaffold.

### Rotatable Housing

The tubular housing enables rotation along its long axis for dispersion of cells during re-seeding or particles during pharmaceuticals or exposure testing. The tubular housing may be transparent, enabling exposure of internal components to light such as in optogenetics experiments, laser-based experiments, or imaging.

### Tissue Sheet Retaining Pieces

There are two types of tissue structures that can be processed. The devices differ depending on which of the two types of tissue structures are to be processed.

The first type of tissues is tubular, such as trachea, blood vessels, bladder, and intestine. These may be secured within the tubular housing as shown in FIGS. 1A-1C.

In another embodiment, the inlet and outlet pieces can include a fitment that enables fixation of one or more tubular organs/tissues/tissue scaffold in the form of a “barb” or different connecting structures that enable the fitment of non-tubular organs/tissues/tissue scaffold. The plug component can have features surrounding or within the fitments that enable fixation of scaffolds or structures outside of the tissue/organ.

The second type of tissue is sheets of tissue or tissue scaffold, which are rolled and secured to form a tube which is connected the same as a tubular structure for processing.

In this second embodiment, the tubular structure is secured to a multi-piece retainer, around which the sheet is rolled to create a tubular structure. The tissue sheet retaining component has a clip to the side that is designed to compress the loose seam of the wrapped sheet-like structure so that the intraluminal fluid does not mix with the extraluminal fluid.

This embodiment is shown in FIGS. 5-7.

FIGS. 5A-5D show the drainage outlet insert 80 for use with flat tissue rolled to form a tubular structure. FIG. 5A is a cross-sectional view of one of the plates 100 located on the end portion of drainage outlet insert 80 which secures one end of the flat tissue structure. Fluid hole 104 drains fluid from the tissue scaffold. FIG. 5B shows the top or inner most plate 100 having holes 102a-h inside rim 104. Tissue clip 108 secures flat tissue into a rolled tubular scaffold. FIG. 5C is a side perspective view, showing plates 100a, 100b, 100c, section 90, expanded section 88, indented section 86, retainer section 84, and end piece 82. Fluid channel 92 runs the length of the drainage outlet insert 80. FIG. 5D is a cross-sectional perspective view.

FIGS. 6A-6B show the inlet plug 106 for use with flat tissue rolled to form a tubular structure. FIG. 6A is a side perspective view; FIG. 6B is a side perspective view. The inlet plug 106 includes exterior section 122 with base rim 124, O-ring recession 118b, separating region 116, O-ring recession 118a, section 114, base support 112, plates 110a, 110b, 110c support prongs 106a-e, and tissue sheet retaining clip 108. It is understood that support prongs 106a-e can be flat, round, rectangular, or other geometry, vary in number, spacing, and height, but must be capable of supporting the flat tissue rolled to form a tubular structure, which is retained in position using clip 108. Fluid channel 124 is shown in FIG. 6A.

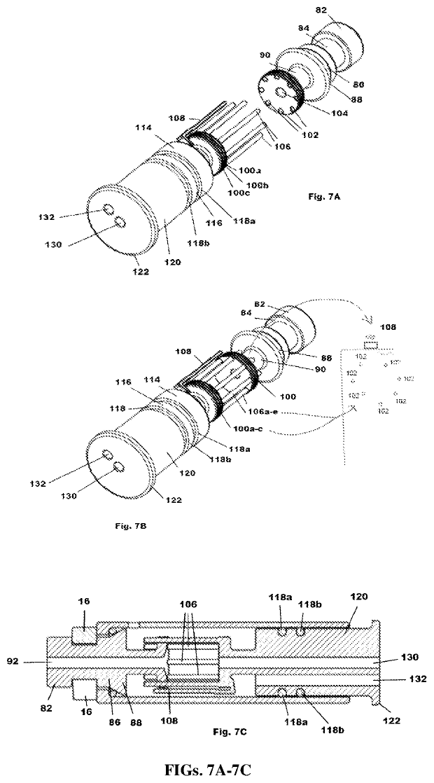

FIGS. 7A-7B show the assembly of the flat tissue bioreactor. FIG. 7A shows the two parts of the flat tissue bioreactor which are fitted together. FIG. 7B shows how support prongs 106a-e fit into the holes of 102a-h on plate 100 of the drainage outlet insert 80. FIG. 7C is a cross-section of the assembled flat tissue bioreactor.

### III. MANUFACTURE AND ASSEMBLY OF COMPONENTS AND TISSUE MANUFACTURE OF MODULAR COMPONENTS

The bioreactor components are preferably formed of a transparent material such as a plastic like an acrylic polymer, polyethylene, polypropylene, polystyrene, polycarbonate, or other similar materials. Glass can also be used. Device components can be formed by extrusion, molding, machining, or a 3-dimensional printing method (3DP, stereolithography). Dimensions and tolerances can be varied using 3D printing techniques and/or SLA printing and FFF printing. The device/bioreactor can be manufactured from optically transparent materials such as various glasses, plastics, and silicone-based materials such as polydimethylsiloxane.

### Assembly of Modular Components

The reactor housing is connected to a source of filtered media and gases using tubing such as TYGON tubing and a manifold for fluid and gas exchange. Pressures are monitored using standard pressure monitors.

Peristaltic pumps are preferably used to move fluid into and out of the reactor because they maintain continuity of flow through tubing and maintain sterility.

The bioreactor is assembled by attaching the “inlet” and “outlet” components to the tissue such as trachea or rolled sheet of tissue and sliding everything into the housing. The assembly is then fixed in the housing with the “clamp”. This enables rapid assembly and fewer parts requiring sterilization. After clamping, the housing is adjusted for organ length by sliding the two pieces of the housing in or out.

### Connection of Tubing and Scaffold

The reactor housing is connected to a source of filtered media and gases using tubing such as TYGON tubing and a manifold for fluid and gas exchange. Pressures are monitored using standard pressure monitors. Peristaltic pumps are preferably used to move fluid into and out of the reactor because they maintain continuity of flow through tubing and maintain sterility.

The bioreactor is assembled by attaching the “inlet” and “outlet” components to the tissue such as trachea or rolled sheet of tissue and sliding everything into the housing. The assembly is then fixed in the housing with the “clamp”. This enables rapid assembly and fewer parts requiring sterilization. After clamping, the housing is adjusted for organ length by sliding the two pieces of the housing in or out.

### IV. METHODS FOR PROCESSING OF TISSUE SCAFFOLDS

### Tissue Scaffolds and Grafts

Many types of tissues can be decellularized and reseeded using the reactor. Preferred embodiments include trachea, heart valves, blood vessels, nerve sleeves, and components of the gastrointestinal tract.

These can be obtained from cadavers, donors, or animals. The advantage of decellularizing the tissue is that this eliminates and/or significantly reduces the immunogenicity, which can then be seeded with cells from the patient in need of the tissue engineered product.

### Selective (Partial) Decellularization

A native tissue or organ graft is fixated between the insert and cap or plug.

Decellularizing agent is introduced to the extraluminal or intraluminal compartments. This may include DNAase or proteinase to ablate native cells.

Culture medium is introduced to the extraluminal or intraluminal compartments to keep native cells viable.

Buffering medium is introduced into the extraluminal or intraluminal compartments for rinsing and dilution of the decellularizing and/or culture medium.

The concentration, duration, and flow rate of the decellularizing agent and culture medium can be varied as needed. The decellularization agent and culture or buffer medium can be alternated to control penetration of decellularizing agent, DNAase, or proteinase

Multiple bioreactors can be utilized in parallel and orientation allowing for rotation of the bioreactors to facilitate fluid and gas distribution. This also helps facilitate cell distribution for even or patterned reseeding or chemical/particle distribution for drug or toxin related experiments. The tubing is connected so that it rotates with the cylinders.

FIG. 8 is a cross-sectional view of the assembled bioreactor containing decellularized trachea 70, connected via valving 80 to peristaltic pumps 82, 84, connected with tubing to SDS 86 flowing to the interior of the trachea 70 and cell culture media DMEM 88 flowing to the exterior of the trachea 70. It is understood that the same type of device could be used with other tissues or organs, and other media, including air.

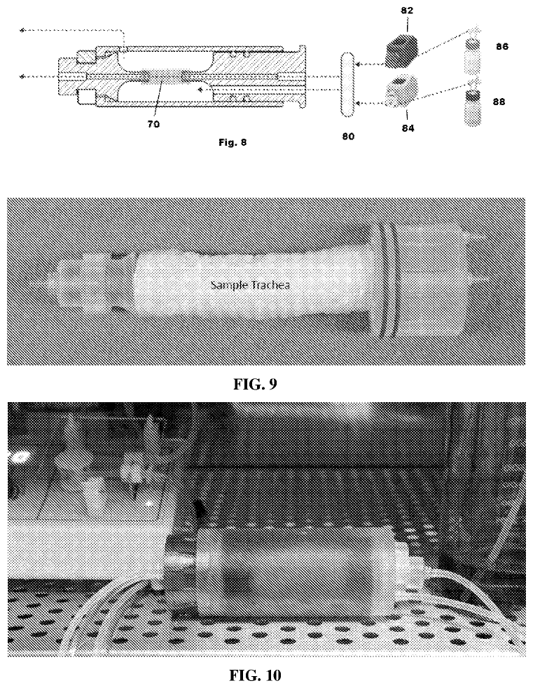

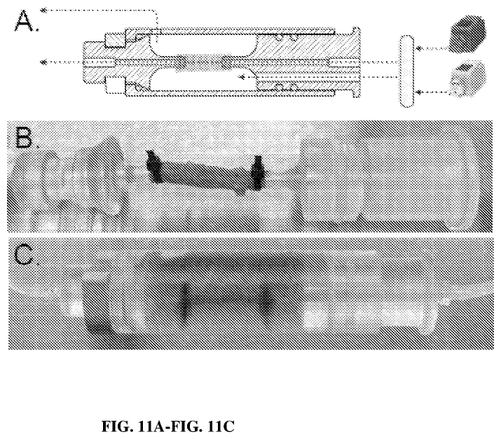

A 5 cm trachea segment was used to test the bioreactor with medium and decellularizing agent. FIGS. 9 and 10 show the assembled and connected bioreactor in use to decellularize and reseed trachea.

### Exemplary Protocol for Full Decellularization:

As shown in FIG. 8, a sheep (O. aries) trachea is isolated and fixated onto the end pieces using cable ties. Tubing from a bottle with 2% SDS in distilled water is connected to the intraluminal and extraluminal compartments of the trachea with silicone tubing. The outlets for the intraluminal and extraluminal compartments are connected to the original 2% SDS bottle. The SDS detergent disrupts cell membranes to fully decellularize the trachea. The SDS is pumped at 50 mL/min using computer-controlled peristaltic pumps for 48 hours or more. The SDS detergent disrupts cell membranes to fully decellularize the trachea. Oxygenation is not important since one is removing, not preserving, the cells.

### Exemplary Protocol for Partial Decellularization:

A rabbit (O. cuniculus) trachea is isolated and fixated onto the end pieces using looped and twisted metal wire, or equivalent. Tubing from a source of 1% SDS in distilled water is connected to a tubing splitter, also connected to a source of DMEM. Dulbecco's Modified Eagle Medium (DMEM) is a widely used basal medium for supporting the growth of many different mammalian cells. DMEM uses a sodium bicarbonate buffer system, to maintain physiological pH in a 5-10% CO₂ environment. The splitter combines both streams into one tube which is connected to a peristaltic pump, which pumps at 5 mL/min. Then this tube is connected to the intraluminal compartment. The intraluminal chamber outlet is connected to tubing that leads to disposal. The DMEM tubing is initially valved shut such that only the SDS solution perfuses the intraluminal chamber. At fixed intervals, the SDS tubing is valved shut and the DMEM tubing valving is open such that the DMEM can expel the SDS. This alternating perfusion is repeated for multiple cycles and various durations to control tissue permeation and accumulation of SDS, which are time dependent. For example, SDS is perfused for 15 minutes and then DMEM is perfused for 5 minutes and this alternating cycle is repeated 12 times to preserve chondrocyte viability while eliminating all other cells in the epithelium and lamina propria. Separate tubing from a bottle of DMEM with Fetal Bovine Serum (FBS) is connected to the extraluminal compartment of the trachea. The DMEM and FBS solution is a culture medium that sustains the viability of the bulk trachea. The outlet for the extraluminal compartment is connected to the original DMEM and FBS bottle. The DMEM and FBS solution is pumped using peristaltic pumps at 5 mL/min. The whole assembly is in an incubator at a temperature of 37 °C, 5% CO₂, and 21% O₂.

### Exemplary Protocol for Reseeding:

A fully decellularized mouse (M. musculus) trachea or artificial graft is isolated and fixated onto the end pieces using looped and twisted metal wire. Tubing connected to a solution bottle of bone marrow-derived mononuclear cells (BM-MNC) in Rosewell Park Memorial Institute (RPMI) medium is connected to the intraluminal compartment. The outlet tubing is connected to the original solution bottle. The BM-MNC solution is perfused 0.5 mL/min or lower using a peristaltic pump. The extraluminal compartment is connected to a stock bottle of RPMI and perfused and recirculated at 1 mL/min. The bioreactor is rotated at 1 degree per minute by computer-controlled motors or servos to enable uniform distribution of BM-MNC. The flow and rotation are maintained for 6 hours for complete reseeding of the internal surface of the graft. The whole assembly is in an incubator at a temperature of 37 °C, 5% CO₂, and 21% O₂.

### Methods of Use for Screening of Agents and Biocompatibility

Although the primary use of these devices is to create tissue grafts or scaffolds, the devices containing the seeded grafts are suitable for screening of compounds for bioactivity, for safety and toxicity, for half-life, for penetration into tissues, and for biocompatibility and non-immunogenicity. For example, the devices containing cells or tissue graft can be exposed to patient cells or leukocytes to test for lack of immune response prior to implantation of the graft into an individual. The graft can also be exposed to suspected irritants and toxins and the resulting immune response or tissue damage assessed. The grafts can be used for screening of new therapeutics, to test for toxicity, efficacy, half-life, and specificity. Rotating the cylinders improves distribution and uniformity of exposure of the cells or tissue to the agents being tested.

The graft can be recellularized using genetically modified cells for optogenetics experimentation, such as for studying organogenesis or disease processes. The graft can be exposed to patterned light of modulated intensity and wavelength for three-dimensional modulation of cell signaling.

The present invention will be further understood by reference to the following non-limiting example.

### Example 1: Preparation of Rabbit Trachea in Bioreactor

### Materials and Methods

### Animal Care and Ethics Statement

The treatment of animals in this study was in accordance with the standards published by the National Institutes of Health (NIH, Bethesda, MD) and regulations defined in the Animal Welfare Act by the United States Department of Agriculture (Washington, D.C.).

### Acquisition of Tracheal Segments

Nine New Zealand white rabbits (Oryctolagus cuniculus, Linnaeus, 1758) between 3.4 to 3.6 kg were euthanized with Euthasol. The airways were resected from the larynx to the carina and then 3.5 cm tracheal segments were collected. Three tracheas were assigned to each of the native, bioreactor-processed, and conventionally-processed groups. The tracheal segments were immersed in phosphate buffered saline with 1% penicillin-streptomycin (P/S, Gibco, Thermo Fisher Scientific, Waltham, MA) for transport prior to processing.

### Bioreactor Fabrication and Setup

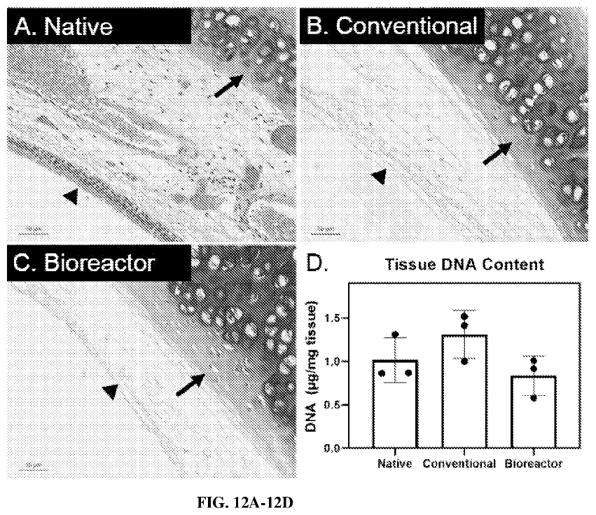

FIGS. 11A-11C shows the bioreactor design and setup: Cross-section of the bioreactor with mock tracheal segment (11A). The dotted arrows represent the intraluminal and extraluminal flow paths. The pumps are represented on the right with the rectangle representing flexible fluid valving and splitting. Rabbit trachea which has been affixed to the two modular end fittings of the bioreactor (11B). Fully installed bioreactor with connected tubing and media flow (11C).

The bioreactor components were printed using a stereolithography printer (Form 3B+ with clear resin, Formlabs, Somerville, MA) at 50 μm resolution with standard isopropanol rinse and post-processing with 395 nm light. The bioreactor is composed of a tube with two end fittings. All components of the bioreactor and tubing were disinfected with 70% ethanol in distilled water prior to use. The fitments were first coupled to the trachea with cable ties. The dimensions of these fitments were retained as an adjustable variable to enable customization based on tracheal diameter. This assembly was then inserted into the bioreactor tube and locked into place with a retaining ring. The fitment which does not lock with the retaining ring is free to be adjusted in or out of the tube to fit various tracheal lengths.

The assembled bioreactor had intraluminal and extraluminal fluid paths which were then connected to peristaltic pumps (Masterflex L/S, VWR, Radnor, PA), tube clamps, and solution bottles using silicone tubing. The intraluminal chamber drew alternatively from two bottles, one containing Dulbecco's Modified Eagle medium (DMEM) and the second containing a 1% solution of the decellularizing detergent sodium dodecyl sulfate (SDS, Sigma-Aldrich, St. Louis, MO), through a flow splitter and two tube clamps. The solution bottles were vented with gas-permeable syringe filters (0.2 μm, Corning, Corning, NY). The extraluminal chamber drew from a bottle containing DMEM with 10% fetal bovine serum (FBS) and 1% P/S. The solutions pumped through both chambers were maintained at a flow rate of 5 mL/minute to minimize shear stress. The bioreactor and solutions were maintained at 37 °C and 5% CO₂ with saturating humidity within a cell culture incubator (Fisherbrand Isotemp, Thermo Fisher Scientific).

### Partial Decellularization Protocols

The conventional partial decellularization protocol uses surfactant and enzymatic activity combined with agitation and osmotic forces for decellularization. Three tracheal segments were subjected to immersion with 1000 rpm agitation in 1% SDS for 3.75 hours, 2% TRITON® X-100 for 2.5 hours, 2000 kU/L DNase in 1M NaCl for 1.25 hours at 37 °C, and double distilled water (ddH₂O) with 1% penicillin-streptomycin for 2.5 hours. These durations were the shortest duration that resulted in complete decellularization of the tracheal epithelium and submucosa. The solutions were maintained at room temperature.

In parallel, three tracheal segments were partially decellularized using the bioreactor. The intraluminal surface of the trachea was exposed to the decellularizing SDS to achieve decellularization of the epithelium and submucosa while the extraluminal surface of the trachea was exposed to culture medium to enable diffusion of nutrients through the sparse adventitia to the cartilage. The intraluminal compartment was exposed to 12 cycles, each cycle consisting of 15 minutes of 1% SDS followed by 5 minutes of DMEM, for a total of 4 hours. The number of cycles was selected because it was the shortest duration necessary for complete de-epithelialization. The extraluminal compartment was exposed to typical culture conditions with a continuous flow of DMEM with 10% FBS and 1% P/S.

### Histology and Imaging

1. The tracheal segments were fixed in 10% formalin, embedded in paraffin, and sectioned at 5 μm for transverse imaging of the trachea. Tissue morphology was assessed with hematoxylin and eosin (H&E) staining (Sigma-Aldrich). The effect of partial decellularization on cartilage extracellular matrix composition was assessed using Masson's trichrome staining. Collagen staining with Masson's trichrome was selected because collagen is the primary structural macromolecule in cartilage.

The images of Masson's trichrome stains were exported as TIFF format to be quantified using the Automated Fibrosis Analysis Tool (AFAT) (Gratz el al Methods X. 2019; 7:22-34. doi:10.1016/j.mex.2019.11.028). The AFAT first filters the images, applies linear regression to identify white pixels from undetermined pixels, and then uses the k-nearest neighbors algorithm to sort whether each pixel represents the blue pixels of Masson's trichrome collagen staining. First, ImageJ (NIH) was used to select a region-of-interest that encompassed the largest continuous section of cartilage and excluded the perichondrium. Next, these images were uploaded into the AFAT to yield the stain percentage

$$( stain percentage = \frac{stained tissue pixels}{stained tissue pixels + non - stained tissue pixels} \times 100 )$$

The pixels categorized as “other” were included in the count of stained tissue since these pixels represented the majority of the hyaline cartilage. Default parameters (5 nearest neighbors and HSV color rules) were used for this analysis.

Chondrocyte viability was evaluated using a live/dead cytotoxicity kit (INVITROGEN®, Thermo Fisher Scientific). The tracheal segments were immersed in Calcium Green-1, AM (6 μl/ml) and ethidium homodimer-1 (10 μl/ml) at room temperature. The tracheal cartilage was exposed with transverse cuts and then imaged with a confocal microscope at 10× magnification (LSM 700, Zeiss, Oberkochen, Germany). Cells with green fluorescence were considered live and cells with red fluorescence were considered dead or damaged cells. Cellular viability was defined as the percentage of living cells out of total cells

$$( viability = \frac{live cells}{live + dead cells} \times 100 )$$

in a representative field of view. ImageJ was used for cell counting.

### DNA Quantification

The extent of tracheal tissue decellularization was quantified using DNA concentration (ng DNA/mg dry tissue). The whole wet tissue was lyophilized overnight and then weighed. The DNA was first extracted with an extraction kit (DNeasy Blood & Tissue Kit, QIAGEN, Germantown, MD) and then measured with a spectrophotometer (Nanodrop™ 2000c, Thermo Fisher Scientific).

### Compression Testing

The radial compressive strength of the tracheal tissue was quantified using uniaxial compression testing with methods adapted from Jones et al. Laryngoscope. 2014; 124(10):2352-2358. doi:10.1002/lary.24739. This testing examines the ability of the trachea to withstand external forces to maintain a patent lumen. A 20 N load cell was attached to a computer-controlled tensile and compression test system (MultiTest 5-I, Mecmesin, West Sussex, United Kingdom). 1 cm tracheal segments were placed sideways on a larger polyethylene block under the load cell assembly such that compression would ultimately cause luminal collapse. 3 mm/minute compression was applied until the lumen was 90%-100% obstructed and the force at 50% luminal obstruction was recorded.

### Statistical Analysis

GraphPad Prism 9 (GraphPad Software Inc., La Jolla, CA) was used for statistical analysis. Tukey's multiple comparison's test was used to test for differences between the native, conventionally-processed, and bioreactor-processed groups. Hypothesis testing results were considered significant if P<0.05. The figures represent data using scatter plots with means and standard deviations.

### Results

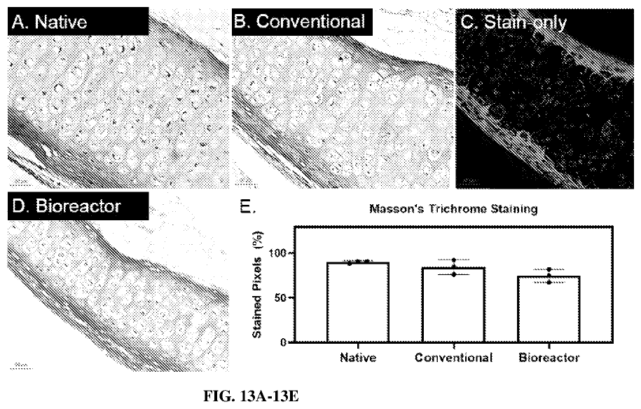

Results are shown in FIGS. 12-14.

### Conventional and Bioreactor Processing Decellularized the Epithelium and Submucosa

The effect of the partial decellularization methods was qualitatively assessed using H&E staining. See FIGS. 12A-12D). Live/dead assay and viability quantification: Representative axial live/dead images of native and processed trachea. (12A-12C). Quantified viability demonstrating that conventional processing results in partially decellularized tracheal grafts with chondrocyte viability lower than that of native and bioreactor-processed trachea (12D). (*** denotes significance with P≤0.001)).

When compared to native trachea, both the conventional immersion and bioreactor processing methods successfully decellularized the epithelium and submucosa, as evident by reduced nuclei and cell bodies, and left the cartilage intact. Next, the tissue DNA content was assessed. Native trachea had a DNA content of 1.02 μg/mg (SD 0.26 μg/mg), conventionally-processed trachea had an average of 1.31 μg/mg (SD 0.27 μg/mg), and bioreactor-processed trachea had an average of 0.84 μg/mg (SD 0.23 μg/mg). There were no differences between these groups (native vs. conventional: P=0.39; native vs. bioreactor: P=0.68; conventional vs. bioreactor: P=0.13).

### Bioreactor Processing Maintained Chondrocyte Viability

A live/dead assay was used to measure chondrocyte viability after partial decellularization.

Native trachea had an average chondrocyte viability of 96.2% (SD 1.9%) which was greater than the viability of 0% (SD 0%) seen in conventionally-processed trachea (P=0.0002). In contrast, bioreactor processing resulted in 79.39% (SD 20.8%) viability which is comparable to native trachea (P=0.28). Bioreactor processing also resulted in better chondrocyte viability compared to conventionally-processed trachea (P=0.0005).

### Conventional and Bioreactor Processing Preserved Cartilage Collagen Content

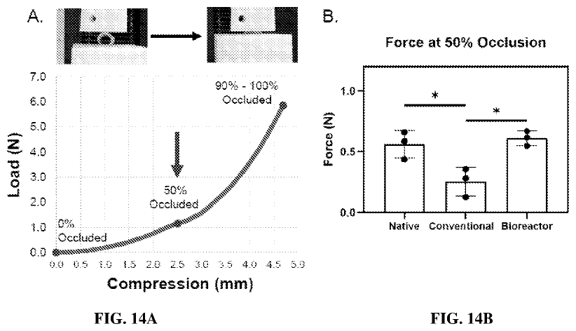

The collagen content of the tracheal cartilage was assessed using Masson's trichrome staining and the AFAT algorithm Representative axial images of native and processed trachea stained with Masson's trichrome are shown in FIGS. 13A, B, D. A representative cropped and stain-only image of (13B) was processed using the AFAT algorithm (13C).

Stain percentages demonstrate no differences between the groups (13E). The AFAT algorithm yielded the approximate percentage of cartilage stained blue by Masson's trichrome staining. Native trachea demonstrated an average of 89.65% (SD 1.39%) staining while conventionally-processed trachea had 84.18% (SD 8.24%) staining and bioreactor-processed trachea had 74.50% (SD 7.27%) staining. The results seen after conventional processing and bioreactor processing did not differ from native trachea (P=0.58 and P=0.062, respectively) and between each other (P=0.23).

### Conventional and Bioreactor Processing Preserved Tracheal Mechanical Strength

FIG. 14A-14B show compression testing method and results: Example load-compression graph with images demonstrating correlated tracheal occlusion. (14A) The arrow shows the datum at 50% occlusion which was used to compare between groups. Force at 50% occlusion which demonstrates that conventional processing results in partially decellularized tracheal grafts that occlude at lower loads when compared to native and bioreactor-processed trachea. (14B) (* denotes significance with P≤0.05)

The force necessary for 50% luminal obstruction was used to compare the mechanical strength of the trachea. The average force resisted by native trachea was 0.56 N (SD 0.11 N), conventionally-processed trachea was 0.26 N (SD 0.12 N), and bioreactor-processed trachea was 0.61 N (SD 0.06 N). The average force measured with the conventionally-processed trachea was lower than that of both native and bioreactor-processed trachea (P=0.02 and P=0.01, respectively). Meanwhile, the average force did not differ between the native and bioreactor-processed trachea (P=0.82).

### DISCUSSION

The 3D printed bioreactor offers a simple four-piece modular design (FIG. 11). The 3D printed components can be rapidly produced at low cost and easily hand-assembled for an accessible and scalable platform that fits a wide range of tracheal dimensions. Pre-existing bioreactors lack these features because they involve complex assembly and lack full adjustability. These features lend to the practicality and flexibility required for clinical research of not only PDTG, but potentially other types of organ grafts. The tube-like design makes it possible to externally rotate the bioreactor which enables uniform delivery of cells for reseeding or other particles for toxicology and pharmaceutical studies. The modular end fittings bring with them almost endless possibilities for culturing any organ or graft geometry. The application we chose here is to produce rabbit PDTG because the bioreactor platform lends to complex processing at human scales.

The platform was validated by generating rabbit PDTG that had chondrocyte viability and collagen content similar to that of native tracheal cartilage while requiring shorter processing times than conventional non-bioreactor processing (FIG. 12, 13). This increased efficiency likely stemmed from the benefits of dynamic flow, which enhanced tissue SDS exposure, SDS replenishment, and removal of cellular debris [26]. Furthermore, using the bioreactor resulted in PDTG that maintained mechanical properties similar to that of native trachea (FIG. 14). The maintenance of these critical characteristics may help contribute to post-transplant cartilage maintenance and airway patency. However, the bioreactor processing did not result in significantly reduced DNA content. This finding demonstrates that tissue DNA content is not a suitable measure for partially decellularized grafts since a population of cells are intentionally preserved. These results show that the bioreactor processing is suitable for efficiently creating PDTG that can be predicted to have favorable long-term airway patency in future transplant studies.

1. A modular bioreactor system comprising
a tubular housing,
an inlet end piece fitting into a first end of the tubular housing, the inlet end piece having an inlet for fluid from the exterior of the end piece through a fluid channel and out the interior of the inlet end piece into the housing, forming an intraluminal compartment, and
an outlet end piece fitting into a second end of the tubular housing, the outlet end piece having an inlet for fluid from the interior of the housing through a fluid channel and out the outlet end piece, forming an extraluminal compartment,
wherein the interior ends of the inlet end piece and the outlet end piece are spaced to allow for insertion of a tissue to be secured to each interior end of the end pieces,
wherein at least one of the end pieces is positionable at different points in the housing to allow adjustment of the spacing between the two interior ends of the inlet end pieces.

2. The modular bioreactor system of claim 1 comprising a first fluid port in the center of each of the inlet and outlet end pieces, and a second fluid port in the outer circumference in each of the inlet and outlet end pieces.

3. The modular bioreactor system of claim 1 comprising fluidic sealing means for the inlet and outlet end pieces in contact with the interior of the tubular housing.

4. The modular bioreactor system of claim 1 comprising a tissue securing end on each of the inlet and outlet end pieces within the tubular housing, wherein the fluid channel passes through the tissue securing ends.

5. The modular bioreactor system of claim 1 further comprising a retainer or clamp to secure one or more of the end pieces within the tubular housing.

6. The modular bioreactor system of claim 1 wherein the outlet end piece comprising fluid sealing means on the exterior of the end piece within the tubular housing to movably secure the end piece within the tubular housing.

7. The modular bioreactor of claim 1 comprising a second fluid channel with one or both of the inlet and outlet end pieces, wherein the second fluid channel is located to the exterior edge of the inlet, and is fluidically directed into the interior of the tubular housing to the exterior of the tissue connecting pieces.

8. The modular bioreactor of claim 7 wherein the tubular housing comprises a fluid port between the interior and exterior of the tubular housing, between the inlet and outlet end pieces.

9. An end piece for a modular bioreactor, the end piece having an exterior end and an interior end, a fluid channel extending from the exterior end through the end piece to the interior end, the end piece comprising a structure for securing a flat sheet of tissue into a tube, the end piece comprising on the circumference of the interior end a multi-prong structure extending away from the end piece, wherein a tissue sheet can be wrapped around and secured to the prongs.

10. A method for recellularization of grafts comprising:
decellularizing in whole or in part tissues or organs secured in the device of claim 1, comprising
delivering of liquid medium containing suspended cells to the intraluminal and extraluminal compartments,
rotating the housing to promote symmetric deposition of cells onto the decellularized tissue or organs.

11. The method of claim 10 comprising introducing decellularizing medium and/or culture medium, then introducing buffering medium into the extraluminal or intraluminal compartments to rinse and/or dilute the decellularizing and/or culture medium.

12. A method for de novo generation of laminar tissues comprising:
fixing a tubular hydrogel or membranous scaffold between the inlet and outlet endpieced in the device of claim 1,
delivering of a liquid medium containing suspended cells to the intraluminal and extraluminal compartments,
sequentially varying medium comprising cells to produce layers of cells.

13. The method of claim 12 comprising flowing media with endothelial cells into the intraluminal compartment and cells such as cells forming extracellular matrix, smooth muscle cells, and/or fibroblasts into the extraluminal compartment.

14. A method for testing of the effect of an agent on tissues and organs formed using the method of claim 10.

15. The method of claim 14 comprising testing a pharmaceutical agent, a potential toxin, gas or smoke on the cells in the graft.

16. A tissue or organ graft formed by the method of claim 10.
